(19) United States
(12) Patent Application Publication
(10) Pub. No.: US 2011/0189702 A1
(43) Pub. Date: Aug. 4, 2011

(54) PHOTOLUMINESCENT MATERIALS FOR MULTIPHOTON IMAGING

(76) Inventor: Ya-Ping Sun, Clemson, SC (US)

(21) Appl. No.: 12/668,212

(22) PCT Filed: Jul. 10, 2008
(86) PCT No.: PCT/US08/69585

§ 371 Date: Nov. 15, 2010

Related U.S. Application Data
(60) Provisional application No. 60/949,070, filed on Jul. 11, 2007.

(51) Int. Cl. G01N 33/53; G01J 1/58; G01N 21/00; G01N 33/00; C08G 73/06; B82Y 30/00

(52) U.S. Cl. 435/7.21; 250/458.1; 436/172; 436/501; 525/417; 250/200; 250/338.1; 977/742; 977/750; 977/752

(57) ABSTRACT

Disclosed are nano-sized materials that can exhibit luminescence in a multi-photon imaging technique. The materials include a nano-sized particle or a carbon nanotube and a passivation agent bound to the surface of the nanoparticle or nanotube. The passivation agent can be, for instance, a polymeric material. The passivation agent can also be derivatized for particular applications. For example, the luminescent materials can be derivatized to recognize and bind to a target material, for instance a biologically active material, a pollutant, or a surface receptor on a tissue or cell surface, such as in a tagging or staining protocol. The materials exhibit strong luminescence with multi-photon excitation in the near infrared.

### CROSS REFERENCE TO RELATED APPLICATION

The present application claims filing benefit of U.S. Provisional Patent Application Ser. No. 60/949,070 having a filing date of Jul. 11, 2007, which is incorporated herein in its entirety.

### FEDERALLY SPONSORED RESEARCH AND DEVELOPMENT

The United States Government may have rights in this disclosure pursuant to grants provided by the Department of Defense Breast Cancer Research Program (grant no. W81XWH-06-1-0656) and the National Science Foundation (grant no. EPS-0132573 and DMR-0243734).

### BACKGROUND

Multi-photon imaging techniques have been suggested as a safer and more accurate method for biological imaging. Multi-photon imaging utilizes luminescent materials that can simultaneously absorb two or more photons to arrive at an excited energy state at which the material can emit a detectable signal in the visible or near-visible spectrum. Multi-photon absorption is possible through focus of a high photon density pulse on the luminescent material. The requisite high photon density is achieved through focusing a high intensity, long wavelength energy pulse on the target as described, for example, in U.S. Pat. No. 5,034,613 to Denk, et al. and U.S. Pat. No. 6,166,385 to Webb, et al. both of which are incorporated herein by reference. Accordingly, the method can utilize long wavelength excitation energy in near-infrared and infrared (IR) spectrum.

Prior to the development of multi-photon imaging techniques, the primary methods available for obtaining suitable excitation of luminescent materials was through the use of high energy radiation such as ultraviolet (UV) light. The high energy excitation source is problematic for biological applications, however, due to the damage done to living cells and other tissue components by UV light. Long wavelength energy, in contrast, is preferable in biological application as it does not lead to the tissue damage caused by UV light.

Even after the development of the technique, which allowed the use of less damaging IR excitation, problems still exist, particularly in the field of biological imaging. For instance, fluorescent and phosphorescent materials that have been used in the technique are often less than desirable for use in conjunction with living tissue. In particular, both the luminescent materials as formed (e.g., fluorescent and phosphorescent dyes and particles) as well as the biological break-down products of luminescent materials raise concerns regarding the use of such materials in conjunction with living tissue and in particular in vivo imaging. For example, good two-photon imaging response has been attained through the use of luminescent nanoparticles based upon heavy metal semi-conductors (e.g., cadmium, indium, germanium, etc.), but these materials are not desirable in biological applications. Moreover, luminescent materials suitable for multi-photon imaging are often quite expensive and their use can dramatically increase the costs associated with any imaging process.

What is needed in the art are materials that can be utilized in a multi-photon imaging process that are biologically compatible and able to be completely and safely expelled from a living system with no harmful effects. What is also needed in the art are low cost luminescent materials that can be utilized in a multi-photon imaging technique.

### SUMMARY

In one embodiment, disclosed herein is a multi-photon method for detecting a material. For example, a method can include focusing an excitation beam including light at a first wavelength on a luminescent material. The first wavelength can be, e.g., in the IR spectrum or the near IR spectrum. The luminescent material can include a carbon-based core structure and a passivation agent on the surface of the carbon-based core structure. The luminescent material can absorb multiple photons (i.e., at least two photons) at the first wavelength and in response emit energy at a second wavelength. This emission can then be detected. More specifically, the detected emission can be at a shorter wavelength, i.e., at higher energy, than the first wavelength of the excitation beam. For example, the second wavelength can be in the visible or the near IR spectra.

The carbon-based core structure can be a particle, e.g., an elongated particle, an amorphous particle, a partial crystalline and/or crystalline particle. In one embodiment, the carbon-based core structure can be a carbon nanotube. In any case, the carbon-based core structure can be formed on a nanometer scale. For instance, the carbon-based core structure can be less than about 20 nm in average diameter. The carbon-based core structure can include additional components, for instance, a magnetic component, in one particular embodiment.

In one embodiment, the surface passivation agent can be a polymer, for example a biopolymer. The surface passivation agent can include reactive functionality, for example a member of a specific binding pair. Accordingly, a method can also include binding the luminescent material to a compound via the reactive functionality. For instance, the luminescent material can be bound to a biologically active compound, e.g., a cell, a tissue, or a pollutant, or a drug or species targeting specific biological receptors, or an antibody.

In another embodiment, the disclosed subject matter is directed to a luminescent material comprising a carbon nanotube and a surface passivation agent bonded to the surface of the carbon nanotube and covering the surface of the carbon nanotube. The surface passivated carbon nanotube is a multi-photon luminescent material. Moreover, the carbon nanotube of the luminescent material can be any carbon nanotube, i.e., either a single walled carbon nanotube or a multi-walled carbon nanotube.

### BRIEF DESCRIPTION OF THE FIGURES

A full and enabling disclosure of the present subject matter, including the best mode thereof, to one of ordinary skill in the art, is set forth more particularly in the remainder of the specification, including reference to the accompanying figures, in which:

FIG. 1 is an atomic force microscopy (AFM) topography image of surface passivated carbon nanoparticles on mica substrate (FIG. 1A), and the height profile along the line in the image (FIG. 1B);

FIGS. 2A and 2B illustrate luminescence images (all scale bars 20 μm) of passivated carbon nanoparticles on glass substrates excited with an argon ion laser at 458 nm (FIG. 2A) and a femtosecond pulsed laser at 800 nm (FIG. 2B);

FIG. 2C is an overlay of FIG. 2A and FIG. 2B;

FIG. 2D shows a closer view of a two-photon image and includes an emission intensity profile along the illustrated line;

FIG. 3 illustrates one-photon (458 nm excitation) and two-photon (800 nm excitation) luminescence spectra of surface passivated carbon nanoparticles located on a glass substrate (prepared with infinite dilution) and compared with solution-phase absorption and luminescence (400 nm excitation) spectra;

FIG. 4 illustrates the quadratic relationship of the observed two-photon luminescence intensity of surface passivated carbon nanoparticles on a glass substrate with the excitation laser power at 800 nm (PExc, as measured at the focal plane);

FIGS. 5A and 5B illustrates representative two-photon luminescence images (800 nm excitation, 20 μm for both scale bars) of human breast cancer MCF-7 cells including internalized surface passivated carbon nanoparticles; and

FIGS. 6A and 6B illustrate luminescence images (all scale bars 3 μm) of functionalized single-walled (FIG. 6A) and multi-walled (FIG. 6B) carbon nanotubes on glass substrates excited with a femtosecond pulsed laser at 800 nm.

### DETAILED DESCRIPTION

Reference will now be made in detail to various embodiments of the disclosed subject matter, one or more examples of which are set forth below. Each embodiment is provided by way of explanation, not limitation, of the subject matter. In fact, it will be apparent to those skilled in the art that various modifications and variations may be made without departing from the scope or spirit of the disclosure. For instance, features illustrated or described as part of one embodiment, may be used in another embodiment to yield a still further embodiment.

The present disclosure is generally directed to nano-sized particulate materials and nanotubes that can be utilized in multi-photon imaging techniques. The disclosed materials can exhibit excellent response during use and can be formed completely of biologically compatible materials. Accordingly, in one preferred embodiment, the disclosed materials are particularly well suited to biomedical imaging processes as they can provide benign alternatives to less ecologically and/or biologically friendly materials, such as those based upon heavy metal semiconductors.

More specifically, the multi-photon imaging materials disclosed herein are composite materials including a carbon-based core structure having a size on the nanoscale (e.g., a carbon nanotube) that is surface passivated with a second material. For purposes of the present disclosure, the term ‘surface passivation’ generally refers to the stabilization or functionalization of the surface of a nanoparticle or a nanotube and is herein defined to include any process in which reactive bonds on the surface of a nanoparticle or a nanotube are terminated and rendered chemically passive. Hence, the term can include elemental passivation, in which a passivating element is bound to an existing bond on a surface, as well as the more generic concept of passivation in which a material can be bound to a surface through formation of a covalent bond between the surface and the material or through noncovalent adsorption, with the possibility of the survival of bonding sites still existing at the surface following the passivation reaction. In this second instance, for example, the passivating material can be a polymer, and the passivation process can form a shell or coating over at least a portion of the surface of a nanoparticle or a nanotube. This shell or coating can be covalently bound to the nanoparticle or nanotube surface at multiple locations, though not necessarily so as to render every reactive bond on the surface chemically passive.

A core nanoparticle can be formed according to any suitable process capable of forming a carbon-based particle on a nanometer scale. For example, in one embodiment, a core carbon nanoparticle can be formed from an amorphous carbon source, such as carbon black; from graphite, for instance in the form of graphite powder; or from crystalline carbon (e.g., diamond). For example, a core carbon nanoparticle can be formed according to a laser ablation method from a graphite starting material. In another embodiment, a core carbon nanoparticle can be formed from carbon powders in an electric arc discharge process. Other methods can be utilized as well, for instance, thermal carbonization of particles of carbon-rich polymers or other precursors. Such methods are generally known to those of ordinary skill in the art and thus are not described in detail herein. Thus, a formed carbon nanoparticle can be amorphous, partial crystalline, or crystalline.

A carbon nanoparticle can generally be any size from about 1 nm to about 100 nm in average diameter. In one embodiment, a core carbon nanoparticle can be less than about 20 nm in average diameter, for instance, in one particular embodiment, between about 1 and about 10 nm in average diameter.

The disclosed materials are not limited to spherical particles. For instance, in other embodiments the nanosized core materials can be multi-faceted, e.g., cubic and the like. In another embodiment, the nanosized materials can be elongated. For instance, the nanosized materials can have an aspect ratio (L/D) greater than 1. According to this embodiment, an elongated nanoparticle can have a diameter in the nanoscale range. For instance, a carbon-based nanoparticle having an aspect ratio greater than 1 for use as disclosed herein can have a diameter less than about 100 nm, or less than about 20 nm, in one embodiment.

Elongated nanoparticles encompassed herein can include carbon nanotubes, including single-walled carbon nanotubes (SWNTs) and/or multiple-walled carbon nanotubes (MWNTs, also including double-walled or DWNTs). The term elongated carbon nanoparticles as utilized herein also encompasses solid carbon-based nanoparticles including, without limitation, carbon fibers, carbon nanowires, and the like. Elongated materials such as carbon nanotubes can exhibit exceptional physical strength, elasticity, high specific surface area, and anisotropic absorption and emission characteristics. In one particular embodiment, the absorption and luminescence of surface-passivated SWNT and MWNT can be polarized along the tube axis.

The methods for producing carbon nanotubes and other elongated carbon nanomaterials are generally known to those of ordinary skill in the art and thus are not described in detail herein. For example, known formation processes for carbon nanotubes include, without limitation, laser ablation methods, chemical vapor deposition methods, electric arc discharge methods, and the like.

In order to attain the ability to exhibit multi-photon luminescence, a passivation agent can be bound to the surface of a carbon nanoparticle or nanotube. A passivation agent can be any material that can bind to a carbon nanoparticle or nanotube surface and encourage or stabilize the radiative recombination of excitons, which is believed to come about through stabilization of the excitation energy ‘traps’ existing at the surface as a result of quantum confinement effects, and the large surface area to volume ratio of a nanoparticle or nanotube. The agent(s) can be bound to a nanoparticle or nanotube surface according to any binding methodology. For example, a passivation agent can bind to a nanoparticle or nanotube surface covalently or noncovalently or a combination of covalently and noncovalently. Moreover, a passivation agent can be polymeric, molecular, biomolecular, or any other material that can passivate a nanoparticle or nanotube surface. For instance, the passivation agent can be a synthetic polymer such as poly(lactic acid) (PLA), poly(ethylene glycol) (PEG), poly(propionylethylenimine-co-ethylenimine) (PPEI-EI), and poly(vinyl alcohol) (PVA). In one embodiment, the passivation agent can be a biopolymer, for instance a protein or peptide. Other exemplary passivation agents can include molecules bearing amino and/or other functional groups.

The passivation agent and/or additional materials grafted to the core nanoparticle or nanotube via the passivation agent (exemplary embodiments of which are discussed in more detail below) can provide the luminescent particles or nanotubes with desirable characteristics, in addition to multi-photon luminescence. For example, a hydrophilic passivation agent can be bound to the core carbon nanoparticle or nanotube to improve the solubility/dispersibility of the nanoparticles or nanotubes in water. In another embodiment, a passivation agent can be selected so as to improve the solubility of the carbon nanoparticle or nanotube in an organic solvent.

In one particular embodiment, the carbon of a core nanoparticle can be amorphous. Due to the presence of localized π electrons and the existence of dangling bonds on amorphous carbon, a passivating material of this particular embodiment can encompass an extremely large number of possible materials. In fact, it is currently believed that a carbon nanoparticle can be passivated and exhibit multi-photon luminescence upon the binding of any material capable of covalently, noncovalently or a combination of covalently and noncovalently bonding at a surface of the nanoparticle. In particular, there is no particular limitation to the type of passivation agents or a surface end group formed according to the passivation reaction.

A core carbon nanoparticle can include other components, in addition to carbon. For example, metals and/or other elements can be embedded in a core carbon nanoparticle. In one particular embodiment, a magnetic metal alone or in combination with other materials, such as, for example, Ni/Y, can be embedded in a core carbon nanoparticle. The addition of materials, e.g., a metal powder, to the carbon nanoparticle can be attained through any process, for instance during the formation process of the carbon particles according to any methods as are generally known to one of ordinary skill in the art. Exemplary methods can include those described in U.S. Patent Application Publication No. 2008/0113448 to Sun, which is incorporated herein in its entirety by reference. Upon the functionalization of such a nanoparticle to provide surface passivation, the resulting luminescent carbon nanoparticle can include the embedded material, e.g., an embedded magnetic metal, and through such can exhibit a desired characteristic, such as magnetic response, which can be useful in many applications including, for example magnetic detection, precipitation and separation, signaling, and the like.

The passivated carbon-based nanomaterials can exhibit multi-photon luminescence when utilized in any multi-photon imaging process as is known in the art. For instance, two-photon imaging protocols have been described in U.S. Pat. No. 5,034,613 to Denk, et al. and U.S. Pat. No. 6,166,385 to Webb, et al, previously incorporated herein by reference. Other two-photon and multi-photon systems and methods that can be utilized in conjunction with the disclosed materials can include, without limitation, U.S. Pat. No. 5,523,573 to Hänninen, et al., U.S. Pat. No. 6,608,716 to Armstrong, et al., and U.S. Pat. No. 6,750,036 to Bearman, et al., all of which are incorporated herein by reference.

For instance, as described by Bearman, et al., multi-photon fluorescence microscopy involves the illumination of a sample with a wavelength around twice the wavelength of the absorption peak of the fluorophore being used. For example, in the case of fluorescein which has an absorption peak around 500 nm, 900 nm excitation could be used. Essentially no excitation of the fluorophore will occur at this wavelength. However, if a high peak-power, pulsed laser is used (so that the mean power levels are moderate and do not damage the specimen), two-photon events will occur at the point of focus. At this point the photon density is sufficiently high that two photons can be absorbed by the fluorophore essentially simultaneously. This is equivalent to a single photon with energy equal to the sum of the two that are absorbed. In this way, fluorophore excitation will only occur at the point of focus (where it is needed) thereby eliminating excitation of the out-of-focus fluorophore and achieving optical sectioning.

The disclosed materials can be comparable in performance to other multi-photon luminescent nanomaterials. For example, the disclosed materials can exhibit two-photon absorption cross section at 800 nm excitation between about 35,000 GM (Goeppert-Mayer unit, with 1 GM=10⁻⁵⁰ cm⁴ s/photon) and about 45,000 GM. As comparison, the two-photon absorption cross-section for CdSe quantum dots at 800 nm varies in the range of 780 GM to 10,300 GM, depending on the particle sizes, as reported in the literature. For CdSe/ZnS core-shell quantum dots (fluorescence at 605 nm), the two-photon absorption cross-section is estimated in the literature reports to be on the order of 50,000 GM.

In one embodiment, multi-photon luminescent materials as described herein can be formed to include a reactive functional chemistry suitable for use in a desired application, e.g., a tagging or analyte recognition protocol. For instance, a passivating agent can include a reactive functionality that can be used directly in a protocol, for example to tag a particular analyte or class of materials that may be found in a sample. Suitable materials can include, for example, carbohydrate molecules that may conjugate with carbohydrates on an analyte or biological species.

In another embodiment, a functional chemistry of a passivation agent can be further derivatized with a particular chemistry suitable for a particular application. For example, in one embodiment, a reactive functionality of a passivating agent can be further derivatized via a secondary surface chemistry functionalization to serve as a binding site for substance. For example, a member of a specific binding pair, i.e., two different molecules where one of the molecules chemically and/or physically binds to a second molecule, such as an antigen or an antibody, can be bound to a nanoparticle or nanotube either directly or indirectly via a functional chemistry of the passivation agent that is retained on the nanoparticle or nanotube following the passivation of the core carbon nanoparticle or nanotube. The passivation and further derivatization of the core carbon nanoparticle or nanotube need not be carried out in separate reactions steps, however, and in one embodiment, the passivation and derivatization of the carbon nanoparticle can be carried out in a single process step.

Accordingly, a luminescent carbon nanoparticle or nanotube can be advantageously utilized to tag, stain or mark materials, including biologically active materials, e.g., drugs, poisons, viruses, antibodies, antigens, proteins, and the like; biological materials themselves, e.g., cells, bacteria, fungi, parasites, etc; as well as environmental materials such as gaseous, liquid, or solid (e.g., particulates) pollutants that may be found in a sample to be analyzed. For example, the passivating material can include or can be derivatized to include functionality specific for surface receptors of bacteria, such as E. coli and L. monocytogenes, for instance. Upon recognition and binding, the bacteria can be clearly discernable due to the photoluminescent tag bound to the surface.

Suitable reactive functionality particular for targeted materials are generally known to those of skill in the art. For example, when considering development of a protocol designed for recognition or tagging of a particular antibody in a fluid sample, suitable ligands for that antibody such as haptens particular to that antibody, complete antigens, epitopes of antigens, and the like can be bound to the polymeric material via the reactive functionality of the passivating material.

Beneficially, the disclosed multi-photon luminescent materials can be more environmentally and biologically compatible than previously known multi-photon luminescent materials. In particular, the disclosed materials can pose little or no environmental or health hazards during use, hazards that exist with many previously known multi-photon luminescent materials. As such, disclosed materials can be utilized in light emission applications, data storage applications such as optical storage mediums, photo-detection applications, luminescent inks, and optical gratings, filters, switches, and the like, just to name a few possible applications as are generally known to those of skill in the art.

In one preferred embodiment, the disclosed materials can be utilized in biomedical imaging. As mentioned above, multi-photon imaging can be preferred in biomedical imaging due to the capability of utilizing long wavelength, near-IR and IR light. Long wavelength light can also be of benefit as an excitation source as it can penetrate deep into tissues, and specifically, deeper than can UV light.

In addition to simply tagging tissue components, such as cells, extracellular matrix components, and the like, the disclosed materials can also exhibit endocytosis and be utilized to image interior components of living cells. While endocytosis has been manifested (see Example 2, below), a complete understanding of the internalization mechanism requires more investigations. In addition, an increased accumulation of nanoparticles in a cell (even in the nucleus) can be achieved, for instance through carbon nanoparticle coupling with membrane translocation peptides such as TAT (a human immunodeficiency virus-derived protein), which can facilitate the translocation of the tissue by overcoming the cellular membrane barrier and can enhance the intracellular labeling efficiency.

The present invention may be better understood by reference to the examples set forth below.

### EXAMPLE 1

Carbon nanoparticles were produced via laser ablation of a graphite powder carbon target in the presence of water vapor (argon was used as the carrier gas, water was deionized and purified by being passed through a Labconco WaterPros water purification system) according to standard methods as described by Y. Suda, et al. (Thin Solid Films, 415, 15 (2002), which is incorporated herein by reference). The as-produced sample contained only nanoscale carbon particles according to results from electron microscopy analyses.

Following an oxidative acid treatment, the particle sample was mixed with poly(propionylethylenimine-co-ethylenimine) (PPEI-EI, EI fraction˜20%) random copolymer, which was obtained via partial hydrolysis of poly(propionylethylenimine) (PPEI, MW˜50,000) (supplied by Aldrich). The mixture was then held at 120° C. with agitation for 72 hours. Following this, the sample was cooled to room temperature and then water was added, followed by centrifuging.

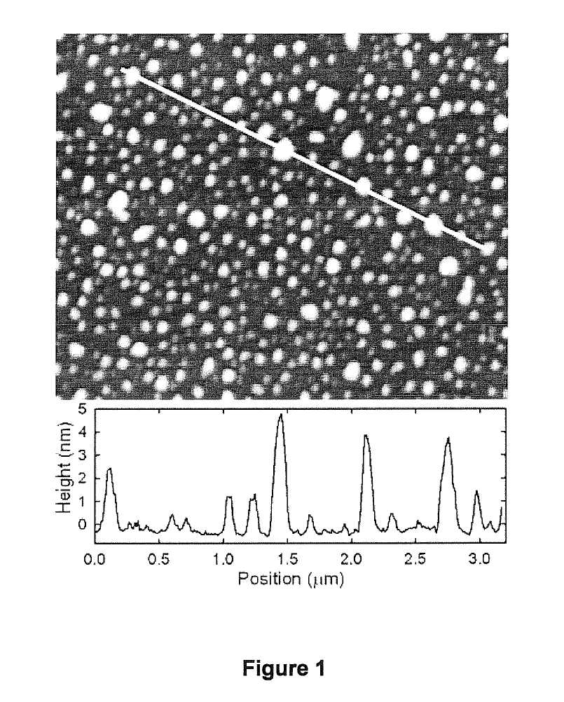

The homogeneous supernatant contained the surface passivated carbon nanoparticles. The nanoparticles thus prepared were readily soluble in water to yield a colored aqueous solution. Shown in FIG. 1A is a representative atomic force microscopy (AFM) image of the surface passivated nanoparticles on mica surface, from which feature sizes of generally less than 5 nm may be identified in the height profile in FIG. 1B.

The nanoparticles were deposited on cover glass by first dropping a small aliquot of their aqueous solution and then evaporating the water. The specimen was analyzed on a Leica confocal fluorescence microscope equipped with an argon ion laser and a femtosecond pulsed Ti:Sapphire laser. An oil immersion objective lens (Leica X63/1.40) was used for confocal and two-photon imaging.

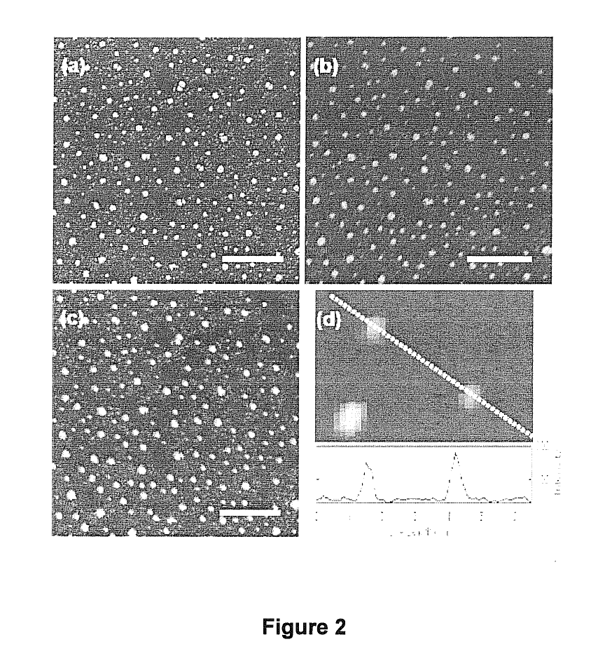

The nanoparticles were found to be strongly emissive in the visible with either the argon ion laser excitation (458 nm) or the femtosecond pulsed laser for two-photon excitation in the near-infrared (800 nm). As compared in FIG. 2, the one- and two-photon luminescence images for the same scanning area match well. Specifically, FIG. 2A illustrates the luminescence using an argon ion laser excitation at 458 nm and FIG. 2B illustrates luminescence using femtosecond pulsed laser excitation at 800 nm. FIG. 2C is an overlay of FIGS. 2A and 2B, and FIG. 2D shows a closer view of a two-photon image with the emission intensity profile taken along the illustrated line.

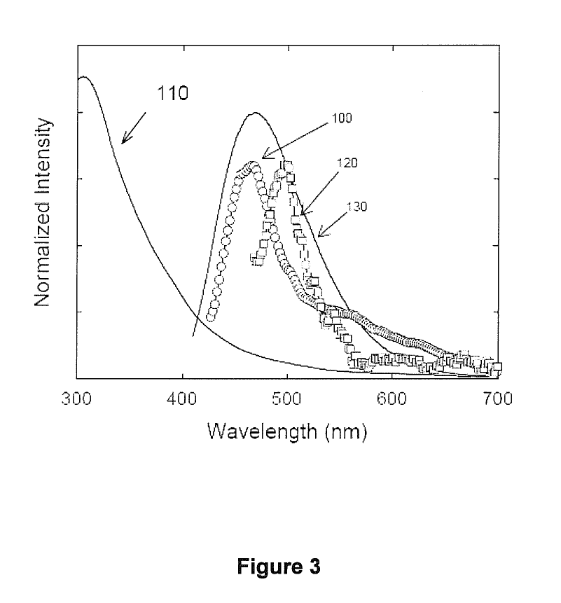

The same optical microscope setup was used to measure two-photon luminescence spectra of the surface passivated carbon nanoparticles on a surface. For the same specimen (surface passivated carbon nanoparticles deposited on cover glass), the observed spectra vary slightly from spot to spot, reflecting the inhomogeneous nature of the sample. A representative two-photon luminescence spectrum of average nanoparticles is shown in FIG. 3 at 100. As can be seen, the two photon luminescence spectrum is narrower in bandwidth than the one-photon solution-phase spectrum excited at 400 nm (absorption at 110 and luminescence at 130). For the nanoparticles on a surface, the same narrow emission bandwidth was observed in the one-photon spectrum at 458 nm excitation (120). These results are again consistent with the inhomogeneity in the sample. Their immobilization on a surface is believed to have allowed the measurement of small fractions in which the emissive species or sites are more homogeneous, corresponding to the narrower luminescence bands for both one- and two-photon excitations.

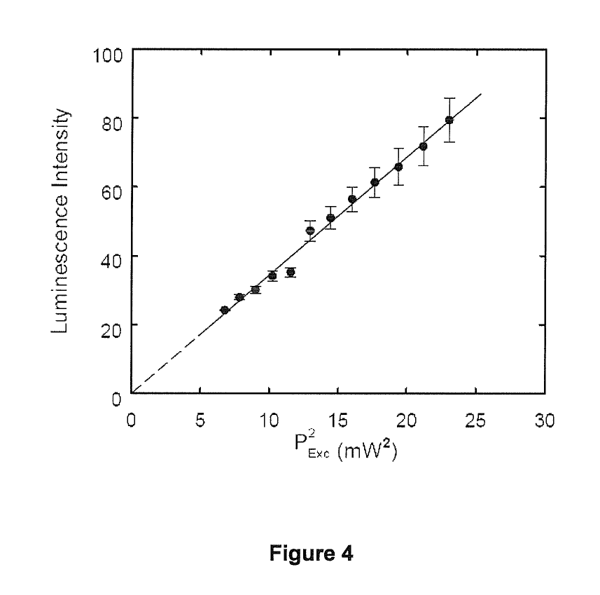

Two-photon luminescence with excitation by femtosecond pulsed laser in the near-infrared was confirmed by the dependence of observed luminescence intensities on the excitation laser power. The luminescence signals were collected with an external detector on the confocal microscope, and the laser powers for excitation were determined by using a precision power meter in the focal plane (thus free from effects of reflection and transmission losses associated with all optical components in the system). As shown in FIG. 4, the quadratic relationship between the excitation laser power and the luminescence intensity is obvious, thus confirming that the excitation with two near-infrared photons was indeed responsible for the observed visible luminescence of the surface passivated carbon nanoparticles.

The two-photon absorption cross-section σ2(λ) of surface passivated carbon nanoparticles was estimated by luminescence measurements of the specimen and a reference compound with the same excitation and other experimental conditions. The two photon absorption cross-section was estimated to be:

σ2(λ)=σ2_{ref}(λ)(<F(t)>/<F_{ref}(t)>)/(φ/φ_{ref})
where
<F(t)> represent averaged luminescence photon fluxes (or experimentally observed emission intensities),
φ are luminescence quantum yields, and
the subscript _{ref} denotes values for the reference compound.

By using rhodamine B as the reference, the two-photon absorption cross-sections of the nanoparticles at different excitation wavelengths were calculated from the experimental results. At 800 nm, the average σ2 value for the nanoparticles was 39,000±5,000 GM (Goeppert-Mayer unit, with 1 GM=10-50 cm⁴ s/photon).

### Imaging Techniques

Atomic force microscopy (AFM) analysis was conducted in the acoustic AC mode on a Molecular Imaging PicoPlus system equipped with a multipurpose scanner for a maximum imaging area of 10 μm×10 μm and a NanoWorld Pointprobe NCH sensor (125 μm in length).

Scanning electron microscopy (SEM) images were obtained on Hitachi 4800 field-emission SEM and HD-2000 STEM systems equipped with energy-dispersive X-ray (EDX) analyzers.

Optical absorption spectra were recorded on a Shimadzu UV3600 spectrophotometer. Solution-phase luminescence spectra were measured on a Spex Fluorolog-2 fluorescence spectrometer equipped with a 450 W xenon source and a detector consisting of a Hamamatsu R928P photomultiplier tube operated at 950 V.

Leica laser scanning confocal fluorescence microscope (DMIRE2, with Leica TCS SP2 SE scanning system) was used for the luminescence imaging and spectral measurements. The microscope is equipped with an argon ion laser (JDS Uniphase) and a femtosecond pulsed (˜100 fs at 80 MHz) Ti:Sapphire laser (Spectra-Physics Tsunami with a 5 W Millennia pump). An oil immersion objective lens (Leica X63/1.40) was used in both one- and two-photon imaging experiments. For the two-photon induced luminescence, an external non-de-scanned detector (NDD) was used for higher signals.

### EXAMPLE 2

Arc-discharge SWNT samples (supplied by Carbon Solutions Inc., or produced in house) were purified according to established procedures (the nitric acid treatment), including the additional use of cross-flow filtration in some purification experiments. No fundamental difference was found in the spectroscopic results with respect to the different sample sources and variations in the sample purification. CVD-produced MWNTs (supplied by Nanostructured & Amorphous Materials, Inc.) were purified also with the established procedure involving nitric acid treatment.

PPEI-EI random copolymer described in Example 1 was used as passivation agent for the nanotube functionalization. The functionalization of SWNTs with PPEI-EI was based on the acylation-amidation of the nanotube-bound carboxylic acid moieties, which are associated with the oxidation of the nanotube surface defects. Experimentally, a purified SWNT sample was refluxed with thionyl chloride for 24 h, followed by evaporation to remove the excess thionyl chloride. To the treated nanotube sample was added carefully dried PPEI-EI, and the mixture was heated to about 170° C. After the reaction for 12 h under nitrogen protection, the mixture was cooled to room temperature. To the mixture was added chloroform, followed by brief sonication and then centrifugation to retain the supernatant. The procedure of extraction with chloroform was repeated multiple times, and the soluble fractions were combined, concentrated, and precipitated into hexane. The PPEI-EI-functionalized SWNT sample was obtained as a dark-colored solid. The sample was characterized by a series of instrumental methods, as reported in the literature. According to the thermogravimetric analysis, the nanotube content in the PPEI-EI-functionalized SWNT sample was on the order of 10% (wt/wt).

Diamine-terminated poly(ethylene glycol) oligomers with molecular structure H2NCH2CH2CH2(OCH2CH2)nCH2NH2 (average n˜35, abbreviated as PEG1500N, supplied by Sigma-Aldrich) was used for the functionalization of MWNTs. A mixture of purified MWNTs and PEG1500N was heated to 120° C. and stirred under nitrogen protection for 4 days. Following reaction, the mixture was cooled to room temperature and then extracted repeatedly with water for the soluble fraction. The combined soluble fraction was cleaned via dialysis, and then evaporated to remove water to yield PEG1500N-functionalized MWNTs. The sample was characterized by a series of instrumental methods, as already reported in the literature.

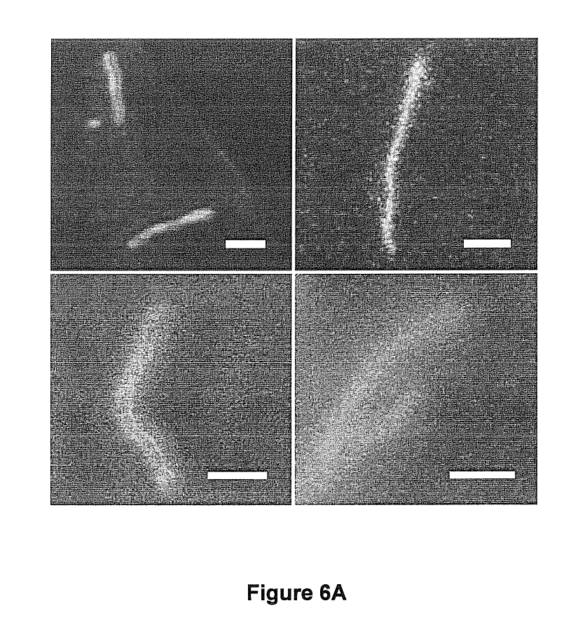

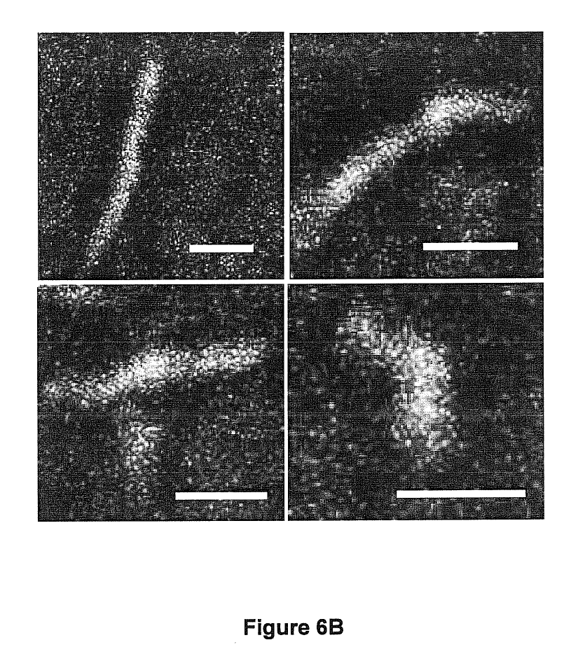

The PPEI-EI-functionalized SWNTs and PEG1500N-functionalized MWNTs were readily soluble in water. The specimens (on cover glass) prepared from aqueous solutions of the two samples were used in the luminescence imaging with a femtosecond pulsed laser at 800 nm to obtain FIG. 6A and FIG. 6B, respectively.

### EXAMPLE 3

The potential of surface passivated carbon nanomaterials for cell imaging with two-photon luminescence microscopy was examined. Human breast cancer MCF-7 cells (approximately 5×105) were seeded in each well of a four-chambered Lab-Tek coverglass system (Nalge Nunc) and cultured at 37° C. All cells were incubated until approximately 80% confluence was reached. Separately, an aqueous solution of passivated carbon nanoparticles formed as described above in Example 1 (0.9 mg/mL) was passed through a 0.2 μm sterile filter membrane (Supor Acrodisc, Gelman Science). The filtered solution (20-40 μL) was mixed with the culture medium (300 μL), and then added to three wells of the glass slide chamber (the fourth well used as a control) in which the MCF-7 cells were grown. After incubation for 2 h, the MCF-7 cells were washed 3 times with PBS (500 μL each time) and kept in PBS for the optical imaging.

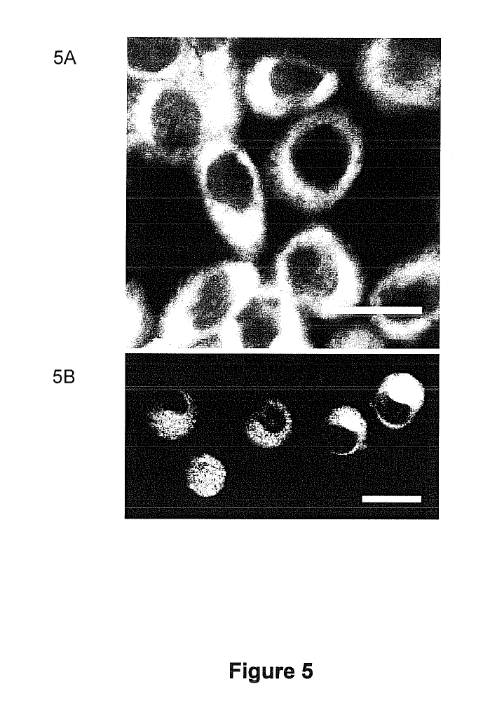

Upon incubation in aqueous buffer at 37° C., the MCF-7 cells became brightly illuminated when imaged on the fluorescence microscope with excitation by 800 nm laser pulses. As shown in FIG. 5, the nanoparticles were able to label both the cell membrane and the cytoplasm of MCF-7 cells without reaching the nucleus in a significant fashion.

The same procedure and conditions were used for the experiment at 4° C. The translocation of the nanoparticles from outside the cell membrane into the cytoplasm was found to be temperature dependent, with no meaningful nanoparticle internalization observed at 4° C.

It will be appreciated that the foregoing examples, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention. Further, it is recognized that many embodiments may be conceived that do not achieve all of the advantages of some embodiments, yet the absence of a particular advantage shall not be construed to necessarily mean that such an embodiment is outside the scope of the present invention.

What is claimed is:

1. A method for detecting a material comprising:
focusing an excitation beam on a luminescent material, the excitation beam comprising light at a first wavelength, the material including a carbon-based core structure and a surface passivation agent on the surface of the carbon-based core structure; and
detecting an emission from the luminescent material, the emission being at a second wavelength that is shorter than the first wavelength; wherein
the luminescent material emits at the second wavelength following absorbance of multiple photons that are at the first wavelength.

2. The method according to claim 1, wherein the carbon-based core structure is a particle.

3. The method according to claim 2, wherein the particle is an elongated particle.

4. The method according to claim 2, wherein the particle is amorphous, partial crystalline or crystalline.

5. The method according to claim 1, wherein the carbon-based core particle is less than about 20 nm in average diameter.

6. The method according to claim 1, wherein the carbon-based core structure is a carbon nanotube.

7. The method according to claim 1, wherein the surface passivation agent is a polymer.

8. The method according to claim 7, wherein the polymer is a biopolymer.

9. The method according to claim 1, the carbon-based core structure further comprising a second component.

10. The method according to claim 9, wherein the second component is magnetic.

11. The method according to claim 1, the surface passivation agent comprising reactive functionality.

12. The method according to claim 11, wherein the reactive functionality is a member of a specific binding pair.

13. The method according to claim 11, further comprising binding the luminescent material to a compound via the reactive functionality.

14. The method according to claim 13, wherein the compound is a biologically active compound.

15. The method according to claim 14, wherein the biologically active compound is a cell.

16. The method according to claim 13, wherein the compound is a pollutant.

17. The method according to claim 13, wherein the compound is a tissue.

18. The method according to claim 1, wherein the first wavelength is in the infrared spectrum or the near infrared spectrum.

19. The method according to claim 1, wherein the second wavelength is in the visible spectrum or near-infrared spectrum.

20. A luminescent material comprising a carbon nanotube and a surface passivation agent bonded to the surface of the carbon nanotube and covering the surface of the carbon nanotube, wherein the surface passivated carbon nanotube is a multi-photon luminescent material.

21. The luminescent material of claim 20, wherein the carbon nanotube is a single walled carbon nanotube.

22. The luminescent material of claim 20, wherein the carbon nanotube is a multi-walled carbon nanotube.

23. The luminescent material of claim 20, further comprising reactive functionality on the surface passivation agent.

24. The luminescent material of claim 23, wherein the reactive functionality is a member of a specific binding pair.

25. The luminescent material of claim 20, wherein the surface passivation agent is a polymer.

26. The luminescent material of claim 25, wherein the polymer is a biopolymer.
